(19) United States
(12) Patent Application Publication
(10) Pub. No.: US 2011/0131711 A1
(43) Pub. Date: Jun. 9, 2011

(54) Convertible Insulating Headcover Apparatus With Flexible Face Shield

(76) Inventors: Marietta Kuchuris, Chicago, IL (US); Dennis M. Sawyer, Chicago, IL (US)

(21) Appl. No.: 12/632,535

(22) Filed: Dec. 7, 2009

(51) Int. Cl. A61F 9/02; A42B 1/06; A42B 1/00

(52) U.S. Cl. 2/426; 2/172; 2/209.11

(57) ABSTRACT

A convertible head cover apparatus for protection against weather elements is disclosed herein. An exemplary embodiment of the apparatus comprises a crown member adapted to be placed on a top portion of a crown of a mammalian head; a flap member pivotally attached to the crown member, the flap member moveable from a stowed position adjacent the crown member to an extended position, the flap member adapted to cover lateral and posterior portions of the mammalian head when the flap member is in the extended position; the crown member having a front flap, the front flap housing a flexible transparent shield member, the front flap being pivotally attached to the crown member and moveable from a stowed position to an extended position, the front flap adapted to cover a portion of a face of the mammalian head when in the extended position; the front flap having a slot forming an opening in the front flap, the transparent shield member removably slideable into and out of the slot to resealably secure the transparent shield member to the front flap when the transparent shield member is inserted into the slot.

### FIELD

This application generally relates to a convertible headgear with at least one flap member and a modular front flap having a removable transparent shield. More particularly, this application relates to a convertible headgear that is designed to flexibly conform to and insulate a substantial part of the face, particularly the eyes, without compromising direct and peripheral lines of sight, and without compromising modular access to the ears or back of the head. The headgear is convertible in that the flexible shield may be raised and stowed, with or without stowing the modular flaps, when not needed. An alternate embodiment substitutes a moveable unibody flap for first, second and rear flaps. In the lowered position, the moveable unibody flap covers not only the ears but also the back of the head. Thus, when both the unibody flap and the moveable crown flap are in the lowered position, the convertible headgear provides 360 degrees of protection from, for example, weather elements, without compromising direct and peripheral lines of sight.

### BACKGROUND

Embodiments of the convertible headgear meet a need because other headgear in the prior art do not completely protect the eyes while permitting unobstructed direct and peripheral lines of sight. Other headgear in the prior art further lack protective and insulating means at the edges of adjoining components. Other headgear also lack the ability to flexibly conform to a wearer's face or accommodate eyeglasses. Prior headgear lack a removable, flexible, transparent shield for unobstructed view or require compromised exposure to the elements at other areas of the head. Thus, embodiments of a modularly protective, convertible headgear apparatus having a flexible, removable, transparent shield remain beyond the contemplation of the prior art.

### SUMMARY

Various embodiments of a convertible headgear having a flexible transparent shield member are described herein. Embodiments disclosed include various combinations of a flexible transparent shield member that are removable and replaceable; a unibody flap member construction; a side flap member construction; and a rear flap member construction. Additional embodiments disclosed include various combinations of construction that provide three hundred sixty degree protection from weather elements and flap member components that are convertible.

### DRAWINGS

While the accompanying claims set forth features of a convertible head cover apparatus with particularity, embodiments of the methods may be best understood from the following detailed description taken in conjunction with the accompanying drawings, of which:

FIG. 1 is a perspective view of an embodiment of a convertible head cover apparatus, comprising a front flap housing a transparent shield member and a flap member, each in a stowed position;

FIG. 2 is a perspective view of an embodiment of the convertible head cover apparatus, wherein the flap member is in an extended position and the front flap is in the stowed position;

FIG. 3 is a perspective view of an embodiment of the convertible head cover apparatus, wherein the flap member and the front flap are each in the extended positions and wherein a releasable connector of the flap member is engaged;

FIG. 4 is a cross-sectional view of the front flap housing a transparent shield member, the front flap having a slot forming an opening in the front flap, the transparent shield member removably slideable into and out of the slot;

FIG. 5 is a lateral view of an embodiment of the convertible head cover apparatus, the apparatus comprising a crown member, wherein the front flap is pivotally attached to the crown member and moveable from the stowed position to the extended position, wherein the transparent shield member reverse-flexes when moved to the stowed position;

FIG. 6 is a lateral view of an embodiment of the convertible head cover apparatus, wherein the transparent shield member flexes concavely when moved to the extended position;

FIG. 7 is a posterior view of an embodiment of the convertible head cover apparatus, wherein the flap member is in the stowed position;

FIG. 8 is a posterior view of an embodiment of the convertible head cover apparatus, wherein the flap member is in the extended position;

FIG. 9 is a posterior view of an embodiment of the convertible head cover apparatus, wherein the flap member is in the extended position;

FIG. 10 is a posterior view of an embodiment of the convertible head cover apparatus, wherein the flap member is in the extended position, wherein the apparatus comprises at least one flap member pivotally attached to the crown member, each flap member moveable from a stowed position adjacent the crown member to an extended position, each flap member adapted to cover at least one of a lateral and posterior portion of the mammalian head in the extended position;

FIG. 11 is a perspective view of an embodiment of the convertible head cover apparatus, wherein the flap member comprises linearly variable attachment points;

FIG. 12 is a perspective view of an embodiment of the convertible head cover apparatus, wherein the front flap comprises at least one tab for linear positioning onto the linearly variable attachment points on the flap member, wherein the flap member and the front flap are adapted to cover a three hundred sixty degree horizontal circumferential extent of the mammalian head below the crown member;

FIG. 13 is a perspective view of an embodiment of the convertible head apparatus wherein the front flap has a slot forming an opening in the front flap, the transparent shield member being removably slideable;

FIG. 13A is a perspective view of an embodiment of the front flap wherein the transparent shield member is being inserted slideably into the front flap with the resealable closure member disengaged;

FIG. 14 is a perspective view of an embodiment of the convertible head cover apparatus, wherein the front flap comprises at least one tab for linear positioning onto the linearly variable attachment points on the flap member, wherein the flap member and the front flap are adapted to cover a three hundred sixty degree horizontal circumferential extent of the mammalian head below the crown member;

FIG. 14A is a perspective view of an embodiment of the front flap wherein the transparent shield member is fully inserted and resealably secured into the slot of the front flap;

FIG. 15 is a perspective view of an embodiment of the convertible head cover apparatus, wherein the flap member comprises first and second opposed extended portions, the extended portions adapted to extend adjacent a chin of the mammalian head when the flap member is in the extended position and a frontal closure member attached to the first extended portion at one end;

FIG. 15A is a perspective view of an embodiment of the convertible head cover apparatus, wherein the first opposed extended portion of the flap member is in a retracted position;

FIG. 15B is a perspective view of an embodiment of the convertible head cover apparatus, wherein the first opposed extended portion of the flap member is both retracted and stowed; and

FIG. 15C is a perspective view of an embodiment of the convertible head cover apparatus, wherein the flap member and the front flap are adapted to cover a three hundred sixty degree horizontal circumferential extent of the mammalian head below the crown member.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

To facilitate illustration of embodiments of the present invention while reviewing the drawings accompanying the specification, a feature table is provided below. Like features are not necessarily like numbered throughout the figures.

FEATURE TABLE

| PART NO. | FEATURE |
|---|---|
| 100 | Convertible head cover apparatus |
| 105 | Stowed position |
| 110 | Lateral portion of a mammalian head |
| 115 | Front flap |
| 120 | Periphery |
| 125 | Transparent shield member |
| 130 | Opening |
| 135 | Flap member |
| 140 | Upper portion of mammalian head |
| 145 | Lower portion of mammalian head |
| 200 | Mammalian face |
| 205 | Insulating edging of front flap |
| 210 | Insulating edging of flap member |
| 215 | Extended position |
| 220 | Linearly variable attachment points |
| 225 | Tab |
| 235 | Releasable connector |
| 240 | Engagement |
| 300 | Crown member |
| 305 | Attachment point |
| 310 | Flexes concavely |
| 315 | Second predetermined length |
| 325 | Flushly |
| 330 | Lower rim |
| 345 | First predetermined length |
| 350 | Three hundred sixty degree horizontal circumferential |
| | extent |
| 400 | Slot |
| 500 | Moveable from an extended position to a stowed position |
| 505 | Two downwardly extending end portions |
| 510 | Flexure |
| 515 | Reverse-flexes |
| 600 | Moveable from a stowed position to an extended position |
| 605 | Thickness |
| 700 | Posterior portion of a mammalian head |
| 800 | Complete length |
| 900 | At least one flap member |
| 1200 | Linear positioning |
| 1210 | Flexibly responsive |
| 1300 | Open position |
| 1305 | Resealable closure member |
| 1400 | Closed position |
| 1405 | Edge |
| 1410 | Seal |
| 1500 | First opposed extended portion |
| 1505 | Frontal closure member |
| 1510 | Second opposed extended portion |
| 1515 | Frontal closure member attachment point |
| 1520 | Reverse-fold configuration |
| 1525 | Air vent |
| 1530 | Fully extended configuration |

Embodiments of a convertible head cover apparatus with flexible transparent shield member are disclosed herein. Embodiments of convertible head cover apparatus 100 may be constructed in part or whole of fur, wool, acrylic, lycra, tricot, spandex, polyester or any other naturally occurring or synthetic materials.

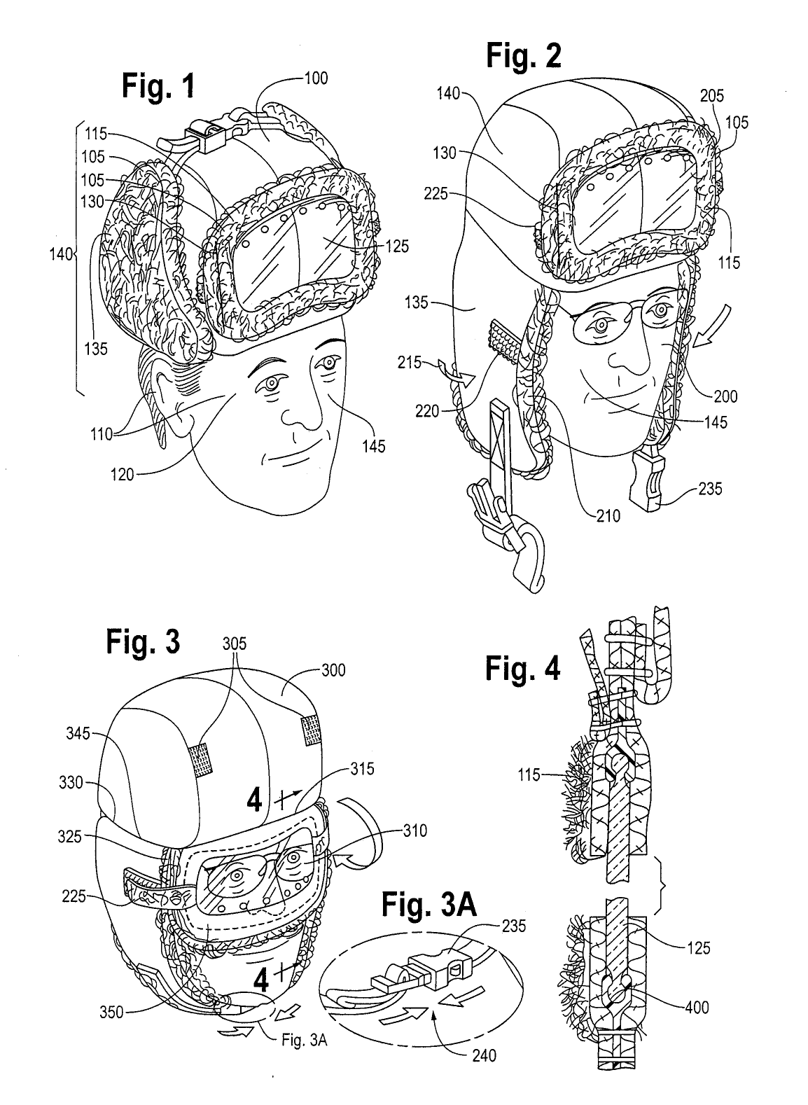

As shown in FIGS. 1, 2 and 3 convertible head cover apparatus 100 is comprised in part of front flap 115, transparent shield member 125, flap member 135, and crown member 300. Apparatus 100 is convertible to protect upper portion of mammalian head 140 and alternatively both lower portion of mammalian head 145 and upper portion of mammalian head 140. As shown in FIGS. 1, 2, 7, 9, 10 and 15, apparatus 100 may further be convertible to protect modular sections of lower portion of mammalian head 145, including periphery 120 of mammalian face 200, lateral portions of mammalian head 110 and posterior portions of mammalian head 700.

As shown in FIG. 1, apparatus 100 may be converted to protect only upper portion of mammalian head 140. In the configuration of FIG. 1, front flap 115 housing transparent shield member 125 is raised and secured to its stowed position 105. Similarly, flap member 135 is also raised and secured to its stowed position 105. As shown in the configuration of FIG. 2, flap member 135 is lowered to extended position 215, while front flap 115 is in stowed position 105. As further shown in the configuration of FIG. 3, both flap member 135 and front flap 115 are lowered to their respective extended positions 215.

One embodiment of transparent shield member 125 is constructed to include polarized plastic sheeting, to reduce glare caused by light reflected from polarizing surfaces such as water as well as by polarized diffuse sky light. An alternate embodiment of transparent shield member 125 is constructed to include mirror coating, deflecting bright light when it hits transparent shield member 125.

As shown in FIG. 2, apparatus 100 may be configured to a modular convertible position in which front flap 115 is raised and secured in a stowed position 105 while flap member 135 is lowered to an extended position 215. In extended position 215 shown in FIG. 2, releasable connector 235 of flap member 135 is not engaged (c.f., FIG. 3A). As shown in this modular configuration, face 200 of lower portion of mammalian head 145 is unprotected. Front flap 115 is comprised of tab 225, used to secure front flap 115 to attachment point 305 (FIGS. 3, 12) to achieve stowed position 105, and used also to secure flap 115 to flap member 135. Opening 130 on front flap 115 is accessible when flap 115 is in its stowed position 105. Also accessible when flap 115 is in its stowed position 105 is insulating edging 205 of front flap 115. Flap member 135 similarly provides its own insulating edging 210 and the two edgings 205 and 210 mate to provide complete insulation from weather elements when both front flap 115 and flap member 135 are each in extended positions 215 and when tab 225 is secured to linearly variable attachment points 220, located on flap member 135. One of skill in the art will appreciate that insulating edgings 205 and 210 may be constructed in part or whole of fur, wool, acrylic, polyester or any other naturally occurring or synthetic materials. One of skill will further appreciate that variable attachment points 220 may be constructed of either a hook or loop fastener. Hook and loop fasteners may be constructed of any suitable material, including without limitation, cotton, nylon, polyester or teflon. In the alternative, a hook and loop fastener may be substituted with a button, clasp, slidingly engaging fastener, single hook and loop, lace or buckle.

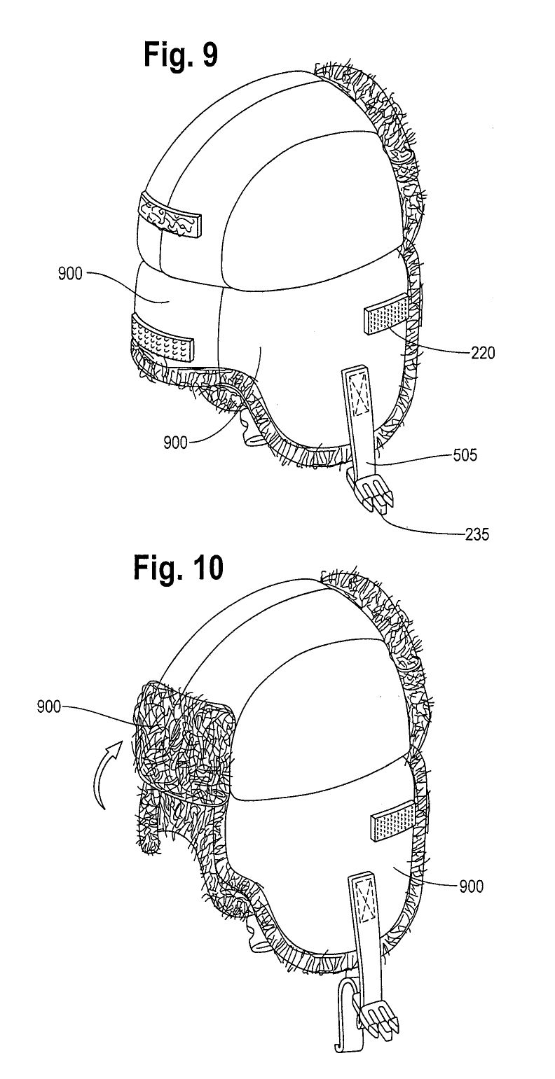

As shown in FIG. 3, apparatus 100 may be converted into a modular configuration in which both front flap 115 and flap members 135 are each in extended positions 215. Crown member 300 is comprised of lower rim 330 and lower rim 330 is comprised of a first predetermined length 345 and a second predetermined length 315. In combination, first and second predetermined lengths 345 and 315 of lower rim 330 comprise a complete length 800 (FIG. 8) of lower rim 330. Flap member 135 is attached to lower rim 330 of crown member 300 along first predetermined length 345 of lower rim 330. Similarly, front flap 115 is attached to lower rim 330 of crown member 300 along second predetermined length 315 of lower rim 330. In this configuration, as discussed above, insulating edgings 205 and 210 are mated flushly 325, thereby providing insulation against external weather elements. When each of edgings 205 and 210 are in extended positions 215 and when tabs 225 are secured to linearly variable attachment points 220 (FIGS. 2, 9), a combination of flap member 135 and front flap 115 cover a three hundred sixty degree horizontal circumferential extent 350 of lower portion of mammalian head 145 below crown member 300. To provide maximum coverage along lower portion of mammalian head 145, flap member 135 is secured into engagement 240 (FIG. 3A) using releasable connector 235 associated with flap member 135.

FIG. 4 shows a cross sectional view of front flap 115 housing transparent shield member 125. In one embodiment transparent shield member 125 is permanently affixed by adhesive to slot 400, sewing or other means known in the art. One of skill in the art will appreciate that any room temperature or thermoplastic adhesive may be used so long as it is suitable for affixing substrates such as transparent shield member 125 to natural and synthetic fabric materials. In another embodiment, transparent shield member 125 is securably removable in and out of slot 400, which forms opening 130 at a subsection of front flap 115. Removability provides a wearer the ability to replace transparent shield member 125 should it become damaged or should optional transparent shield members with tinting, polarization or other treated features be desired. One of skill in the art will appreciate that removing transparent shield member 125 also facilitates the storage, washing or dry cleaning of apparatus 100.

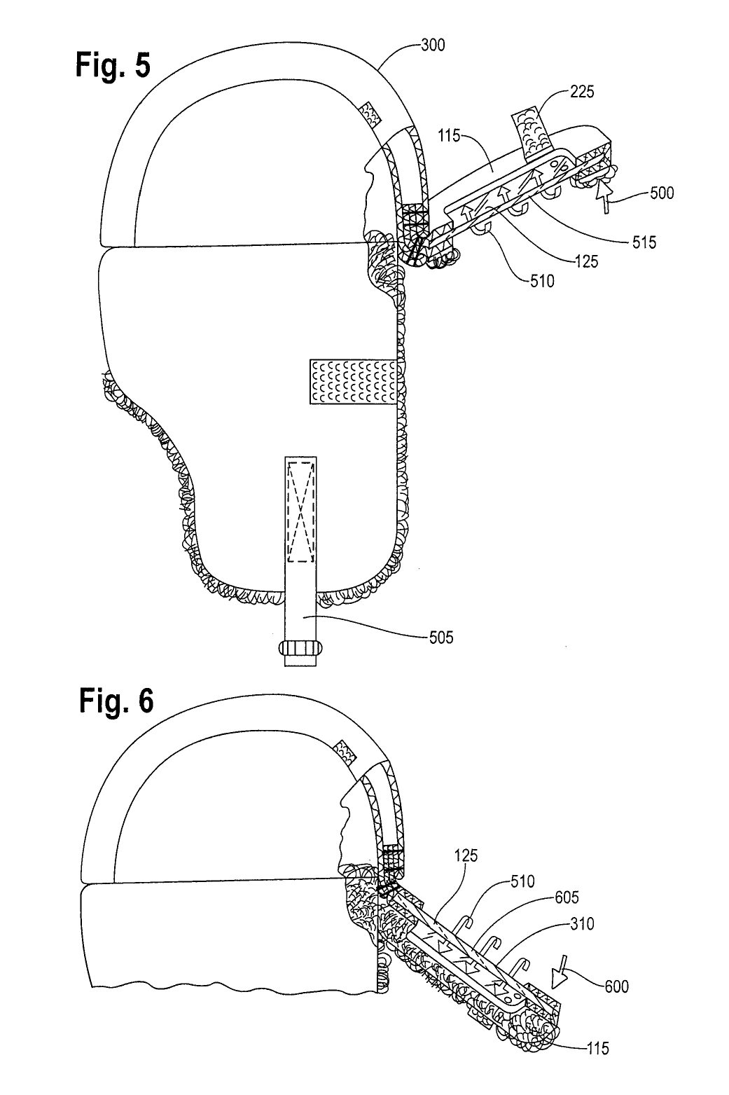

As shown in FIG. 5, front flap 115 is moveable 500 from an extended position to a stowed position. To accomplish this, front flap 115 is pivotally attached to crown member 300. As discussed above, front flap 115 houses transparent shield member 125. When front flap 115 is moved from stowed position 105 (FIG. 1) to extended position 215 (FIG. 2), transparent shield member 125 flexes in one direction. Conversely, when front flap 115 is moved 500 from extended position 215 to stowed position 105, transparent shield member 125 flexes in an opposed direction. Such flexure 510 flexibly adapts transparent shield member 125 to substantially conform to the wearer's unique head size, whether front flap 115 is in stowed position 105 or extended position 215. As shown in FIG. 5, transparent shield member 125 reverse-flexes 515 when moved 500 to stowed position 105. Flap member 135 comprising two downwardly extending end portions 505 allows for substantial conformance of flap member 135 to the wearer's head size by engagement 240 of connector 235 (FIG. 2, 3A). Such flexure 510 flexibly adapts transparent shield member 125 to substantially conform to the wearer's face when in extended position 215.

As shown in FIG. 6, transparent shield member 125 flexes concavely 310 (see also FIG. 3) when moved 600 from a stowed position to an extended position 215, accommodating varying nose sizes and optionally eyeglasses as well. Embodiments of transparent shield member 125 are both flexible and constructed of polyethylene, having a thickness 605 in the range of about 0.5 to 6 mm. For example, one alternate embodiment of transparent shield member 125 will be flexible, constructed of polyethylene and have a thickness 605 of about 2.5 mm. One of skill will appreciate that the construction of transparent shield member 125 need not be limited to polyethylene or polyethylene-derived material, but can be constructed of any other suitable transparent material so long as it flexes concavely 310 to conform to lower portion of mammalian head 145 when in extended position 215.

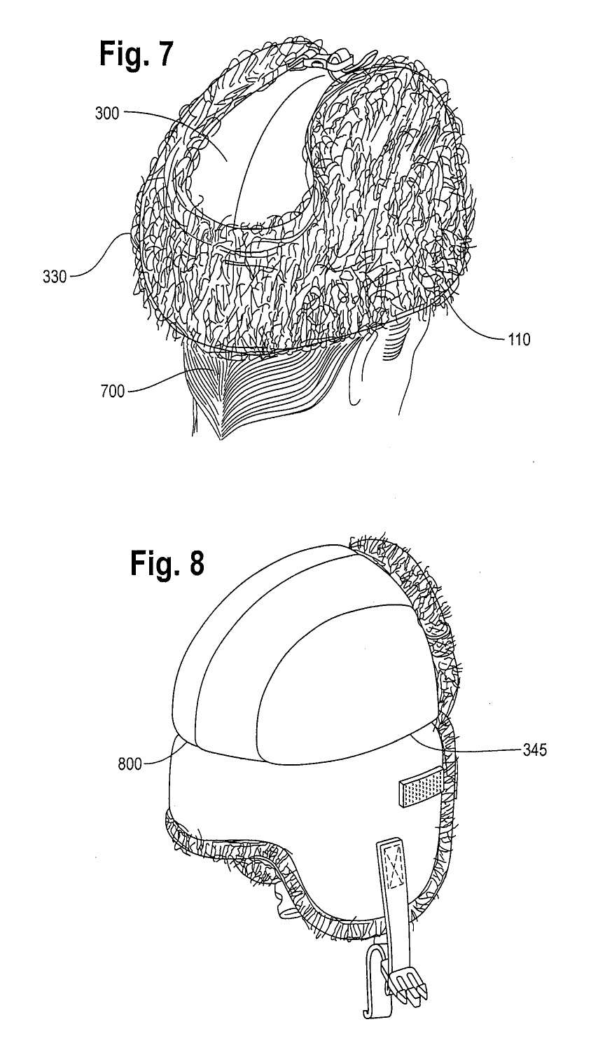

As shown in FIGS. 7 and 8, flap member 135 is in its stowed position 105 (FIG. 1) and its extended position 215 (FIG. 2), respectively. In this embodiment of apparatus 100, flap member 135 is pivotally attached to crown member 300 at lower rim 330 as one contiguous flap. Therefore, when flap member is lowered to its extended position 215 flap member 135 protects both posterior portion of mammalian head 700 and lateral portion of mammalian head 110.

FIGS. 9-10 show an alternate embodiment of apparatus 100. In FIG. 9, a first and a second at least one flap member 900 is shown. Here, both the first and second at least one flap members 900 are in their respective extended positions 215. In FIG. 10, a first and a second at least one flap member 900 is shown. Here, first at least one flap member 900 is in its extended position 215 while second at least one flap member 900 is in its stowed position 105. In this embodiment, moving at least one flap member 900 into its extended position 215 will cover a predetermined modular section of lower portion of mammalian head 145. In this embodiment at least one flap 900 can be configured to modularly cover one or both of posterior portion of a mammalian head 700 and lateral portion of a mammalian head 110. In this embodiment, the wearer can gain access to one or both ears without removing apparatus 100 to use, for example, a telephone or earphone.

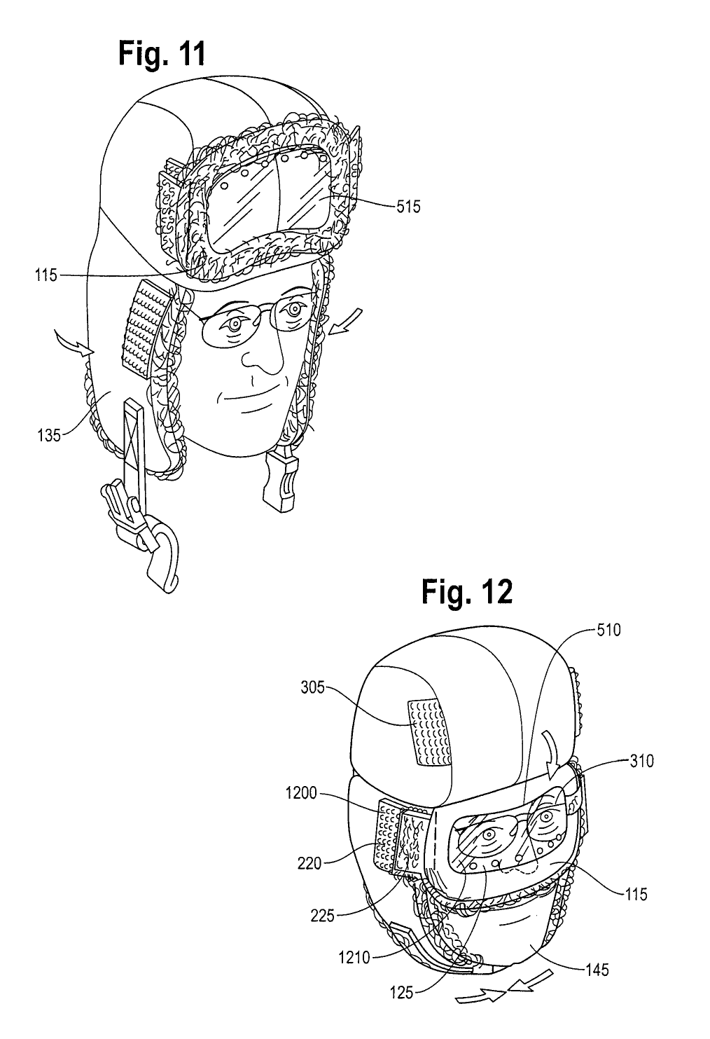

As shown in FIGS. 11-12, one embodiment of apparatus 100 includes an alternate embodiment of tab 225, including attachment means, such as a loop fastener. As shown, tab 225 is elongated, extending vertically in the direction of the length of an extended flap member 135 while in its extended position 215. Tab 225 covers over the mating of insulating edging 205 of front flap 115 and insulating edging 210 of flap member 135.

As shown in FIG. 12, both front flap 115 and flap member 135 are each in extended positions 215. In this configuration, transparent shield member 125 (housed within front flap 115) provides flexure 510 so that front flap 115 flexes concavely 310 when moved to extended position 215. Tab 225 can be moved for linear positioning 1200 onto linearly variable attachment points 220 that are affixed to flap member 135. Transparent shield member 125 housed in front flap 115 flexibly responds 1210 as tab 225 is positioned linearly 1200 onto variable attachment points 220. The more linearly 1200 tab 225 is pulled toward the posterior portion of mammalian head 700, the greater flexure 510 of transparent shield member 125. In this embodiment, flexure 510 of transparent shield member 125 can vary from approximately 1 to approximately 60 degrees, depending in part on the relative positioning of tab 225 on linearly variable attachment point points 220 (or attachment points 305 when reverse-flexed 515 in stowed position 105). Additional factors determinative of degree of flexure 510 include the relative size of apparatus 100, the relative size of mammalian face 200, the relative size of mammalian head portions 110, 700, 140 and 145, including the wearer's nose size, and whether, for example, the wearer is wearing eyeglasses or goggles. In the extended configuration of FIG. 12, when tab 225 is attached to linearly variable attachment points 220, front flap 115 and flap member 135 cover a three hundred sixty degree horizontal circumferential extent 350 of a mammalian head below crown member 300.

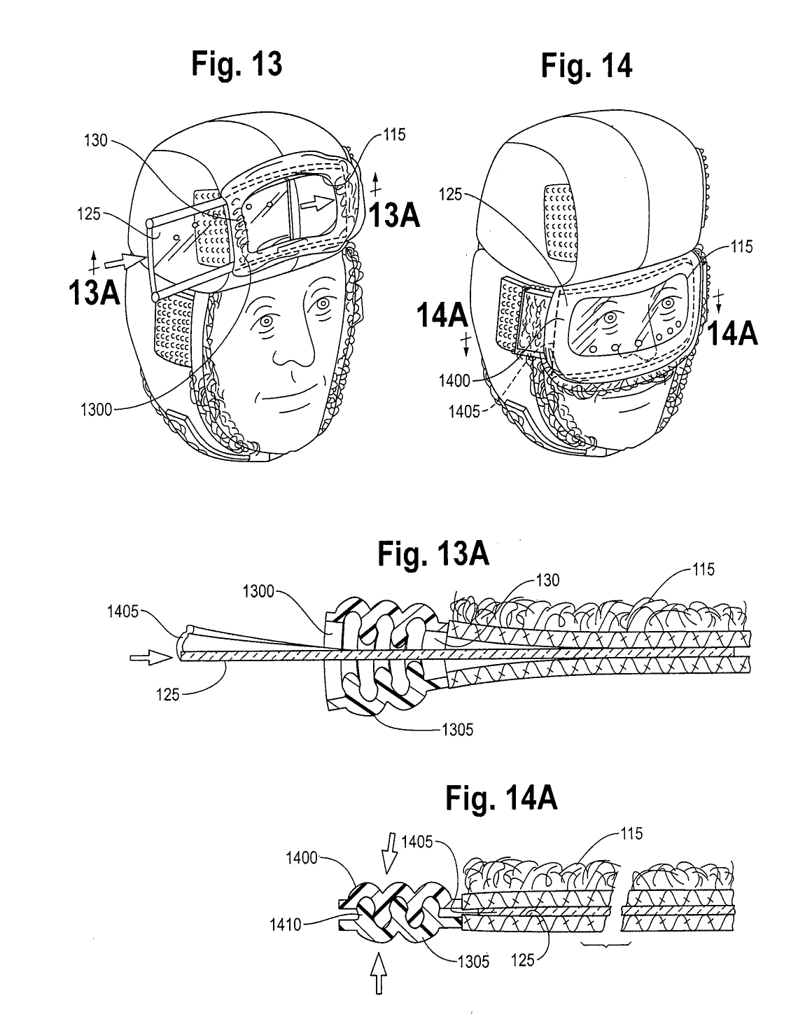

Referring now to FIGS. 13 and 13A, when in open position 1300, front flap 115 forms opening 130 and houses slot 400 (FIG. 4) in front flap 115. Transparent shield member 125 is housed within front flap 115 and is removably slideable into and out of slot 400 to resealably secure transparent shield member 125 to front flap 115 when transparent shield member 125 is inserted into slot 400. As shown in FIG. 13A, an embodiment of front flap 115 provides resealable closure member 1305 for resealably securing transparent shield member 125 at its edge 1405. Slot 400 provides opening 130 formed by resealable closure member 1305 of front flap 115. Resealable closure member 1305 provides an open position 1300 allowing transparent shield member 125 to be inserted into slot 400.

As shown in FIGS. 14 and 14A, when in closed position 1400, front flap 115 completely houses and removably secures transparent shield member 125. To resealably secure transparent shield member 125 in slot 400, resealable closure member 1305 when in closed position 1400 forms a seal 1410 with edge 1405 of transparent shield member 125.

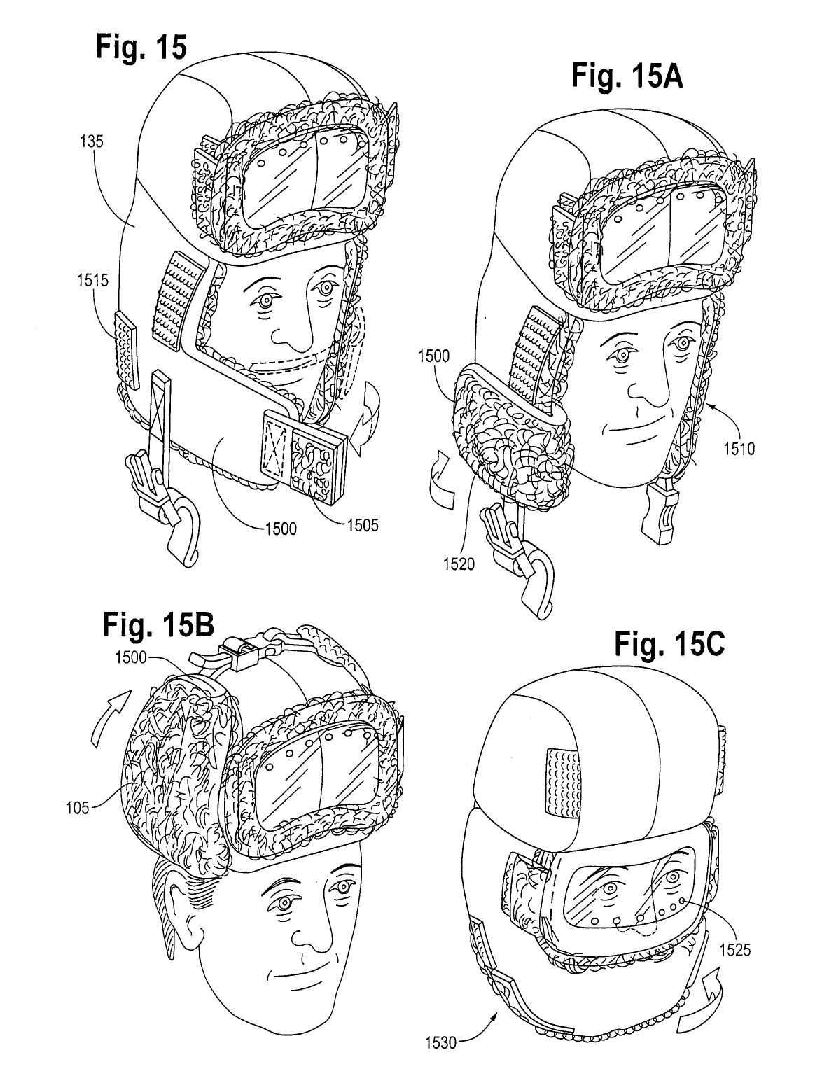

As shown in FIGS. 15-15C, embodiments of convertible head cover apparatus 100 include flap member 135 having first opposed extended portion 1500 and second opposed extended portion 1510, each adapted to extend adjacent a chin of a mammalian head when in extended position 215. First opposed extended portion 1500 includes frontal closure member 1505 attached at one end. Frontal closure member 1505 can thus be removably attached by any known means to second opposed extended portion 1510. When apparatus 100 is worn and frontal closure member 1505 attaches first and second opposed extended portions 1500 and 1510, respectively, flap member 135 covers virtually all of lower portion of mammalian head 145, including the mouth and chin.

As best seen in FIG. 15A, first opposed extended portion 1500 can be folded in reverse and attached to frontal closure member attachment point 1515 using frontal closure member 1505. In this reverse-fold configuration 1520, though flap member 135 is extended, first opposed extended portion 1500 is secured but not extended to cover the chin and mouth sections of lower portion of mammalian head 145.

As best seen in FIG. 15B, reverse-fold configuration 1520 of first opposed extended portion 1500 permits movement of flap member 135 from extended position 215 to stowed position 105.

As shown in FIG. 15C, when front flap 115 is in extended position 215, and flap member 135 is in extended position 215 while frontal closure member 1505 is removably attached to second opposed extended portion 1510, a three hundred sixty degree horizontal circumferential extent 350 covers lateral 110, posterior 700, upper 140 and lower 145 portions of the mammalian head, including periphery 120 of mammalian face 200. Also shown in FIG. 15C, air vent 1525 provides air ventilation when apparatus 100 is in its fully extended configuration 1530.

Embodiments of the present invention may be configured in other specific forms without departing from its spirit or essential characteristics. It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled. While the foregoing description of embodiments of the convertible head cover apparatus 100 may contain many specificities, these specifics should not be construed as limitations on the scope of the convertible head cover apparatus 100 set forth above, but rather as an exemplification of several embodiments thereof.

Claimed is:

1. A convertible head cover apparatus, comprising:
a crown member adapted to be placed on a top portion of a crown of a mammalian head;
a flap member pivotally attached to said crown member, said flap member moveable from a stowed position adjacent said crown member to an extended position, said flap member adapted to cover lateral and posterior portions of said mammalian head when said flap member is in said extended position;
said crown member having a front flap, said front flap housing a flexible transparent shield member, said front flap being pivotally attached to said crown member and moveable from a stowed position to an extended position, said front flap adapted to cover a portion of a face of said mammalian head when in said extended position; and
said front flap having a slot forming an opening in said front flap, said transparent shield member removably slideable into and out of said slot to resealably secure said transparent shield member to said front flap when said transparent shield member is inserted into said slot.

2. A convertible head cover apparatus according to claim 1, wherein said transparent shield member housed within said front flap flexes concavely when moved to said extended position.

3. A convertible head cover apparatus according to claim 1, wherein said transparent shield member comprises at least one air vent.

4. A convertible head cover apparatus according to claim 1, wherein said slot having an opening formed by a resealable closure member of said front flap, said resealable closure member having an open position allowing said transparent shield member to be inserted into said slot, said resealable closure member having a closed position forming a seal with an edge of said transparent shield.

5. A convertible head cover apparatus according to claim 1, wherein said transparent shield member reverse-flexes when moved to said stowed position.

6. A convertible head cover apparatus according to claim 1, wherein said transparent shield member is removable from said front flap.

7. A convertible head cover apparatus according to claim 1, wherein said flap member is attached to a lower rim of said crown member along a first predetermined length of said lower rim; wherein said front flap is attached to said lower rim of said crown member along a second predetermined length of said lower rim; wherein said first and second predetermined lengths of said lower rim comprising a complete length of said lower rim; wherein said flap member and said front flap are adapted to cover a three hundred sixty degree horizontal circumferential extent of said mammalian head below said crown member.

8. A convertible head cover apparatus according to claim 1, wherein said transparent shield member is comprised of polyethylene.

9. A convertible head cover apparatus according to claim 1, wherein at least a portion of said transparent shield member is polarized.

10. A convertible head cover apparatus according to claim 1, wherein at least a portion of said transparent shield member deflects ultra violet light.

11. A convertible head cover apparatus according to claim 1, wherein at least a portion of said transparent shield member is scratch resistant.

12. A convertible head cover apparatus according to claim 1, wherein said front flap in said extended position attaches flushly with said flap member when said flap member is in said extended position.

13. A convertible head cover apparatus according to claim 1, wherein said flap member is adapted to substantially cover a temple of said lateral portion of said mammalian head when said flap member is in said extended position.

14. A convertible head cover apparatus according to claim 1, wherein said flap member comprises first and second opposed extended portions, said extended portions adapted to extend adjacent a chin of said mammalian head when said flap member is in said extended position; and a frontal closure member attached to said first extended portion at one end, said frontal closure member being removably attached to said second extended portion when said cover apparatus is worn on said mammalian head adapted to extend over said chin of said mammalian head when said frontal closure is attached to said second portion.

15. A convertible head cover apparatus according to claim 1, wherein said front flap in said extended position is adapted to insulate a periphery of said face of said mammalian head.

16. A convertible head cover apparatus according to claim 1, wherein an insulating edging of said front flap mates with an insulating edging of said flap member when said flap member and said front flap are both in said extended positions.

17. A convertible head cover apparatus according to claim 1, wherein said front flap further comprises at least one tab, wherein said at least one tab removably attaches to said flap member when said flap member and said front flap are both in said extended positions.

18. A convertible head cover apparatus according to claim 17, wherein said at least one tab removably attaches to said crown member to maintain said front flap in said stowed position.

19. A convertible head cover apparatus according to claim 14, wherein said frontal closure member folds back to attach to a frontal closure member attachment point located on said flap member.

20. A convertible head cover apparatus according to claim 1, wherein said front flap in said stowed position is removably attached to said crown member.

21. A convertible head cover apparatus according to claim 1, wherein said front flap in said extended position is removably attached to said flap member in said extended position.

22. A convertible head cover apparatus according to claim 1, wherein said flap member comprises linearly variable attachment points for removable attachment of said front flap to said flap member when said front flap and said flap member are each in said extended position.

23. A convertible head cover apparatus according to claim 22, wherein said linearly variable attachment points on said flap member flexibly conform to said mammalian face.

24. A convertible head cover apparatus according to claim 22, wherein said linearly variable attachment points on said flap member flexibly conform to said mammalian face to accommodate glasses and alternatively goggles.

25. A convertible head cover apparatus according to claim 22, wherein said transparent shield member housed by said front flap being flexibly responsive to said linear positioning of said attachment points causes flexure of said transparent shield member.

26. A convertible head cover apparatus according to claim 1, wherein said flap member comprises two downwardly extending end portions, said end portions adapted to extend adjacent a lower portion of said mammalian head.

27. A convertible head cover apparatus according to claim 26, wherein said flap member comprises a releasable connector holding one of said end portions in contact with said other end portion upon engagement of said connector.

28. A convertible head cover apparatus according to claim 27, wherein said releasable connector maintains said flap member in said extended position when said end portions of said flap member are connected by said connector.

29. A convertible head cover apparatus, comprising:
a crown member adapted to cover a top portion of a crown of a mammalian head;
at least one flap member pivotally attached to said crown member, each said flap member moveable from a stowed position adjacent said crown member to an extended position, each said flap member adapted to cover at least one of a lateral and posterior portion of said mammalian head in said extended position;
each said flap member independently attached to said crown member, each said flap member moveable between said stowed position and said extended position independent of movement of another flap member;
said crown member having a front flap, said front flap housing a transparent shield member, said front flap being pivotally attached to said crown member and moveable from a stowed position to an extended position, said front flap adapted to cover a portion of a face of said mammalian head when in said extended position;
said transparent shield member flexing when said front flap moves between said stowed position and said extended position.

30. A convertible head cover apparatus according to claim 29, wherein said transparent shield member flexes concavely when said front flap is moved to said extended position.

31. A convertible head cover apparatus according to claim 29, wherein said at least one flap member comprises two flap members, each said flap member having first and second opposed extended portions.

32. A convertible head cover apparatus according to claim 29, wherein said transparent shield member reverse-flexes when said front flap is moved to said stowed position.

33. A convertible head cover apparatus according to claim 29, wherein each said flap member comprises linearly variable attachment points for removable attachment of said front flap to each said flap member when said front flap and each said flap member is each in said extended position.

34. A convertible head cover apparatus according to claim 29 wherein each said flap member is adapted to substantially cover a temple of said lateral portion of said mammalian head when each said flap member is in said extended position.

35. A convertible head cover apparatus according to claim 29, wherein each said flap member comprises first and second opposed extended portions, said extended portions adapted to extend adjacent a chin of said mammalian head when each said flap member is in said extended position; and a frontal closure member attached to said first extended portion at one end, said frontal closure member being removably attached to said second extended portion when said cover apparatus is worn on said mammalian head adapted to extend over said chin of said mammalian head when said frontal closure is attached to said second portion.

36. A convertible head cover apparatus according to claim 31, wherein each said flap member comprises a frontal closure member attachment point for removable attachment of said first opposed extended portion of each said flap member when each said flap member is in said extended position.

37. A convertible head cover apparatus according to claim 36, wherein said frontal closure member folds to attach to said frontal closure member attachment point located on said flap member in a reverse-folded configuration to allow said flap member to be moved from said extended position to said stowed position.

38. A convertible head cover apparatus according to claim 29, wherein said transparent shield member housed by said front flap being flexibly responsive to said linear positioning of said attachment points causes flexure of said transparent shield member.

39. A convertible head cover apparatus according to claim 29, wherein said transparent shield member flexes concavely to substantially conform to a shape of said face of said mammalian head when said front flap is removably attached to each said flap member when each said front flap and each said flap member is each in said extended position.

40. A convertible head cover apparatus according to claim 29, wherein said transparent shield member reverse-flexes to conform to a shape of said crown member when said front flap is moved to said stowed position.

41. A convertible head cover apparatus according to claim 29, wherein each said flap member is attached to a lower rim of said crown member along a first predetermined length of said lower rim;
wherein said front flap is attached to said lower rim of said crown member along a second predetermined length of said lower rim;
wherein said first and second predetermined lengths of said lower rim comprise a complete length of said lower rim; and
wherein a combination of each said flap member and said front flap are adapted to cover a three hundred sixty degree horizontal circumferential extent of said mammalian head below said crown member.

42. A convertible head cover apparatus according to claim 29, wherein said transparent shield member is comprised of polyethylene.

43. A convertible head cover apparatus according to claim 29, wherein said transparent shield member resists scratching.

44. A convertible head cover apparatus according to claim 29, wherein at least a portion of said transparent shield member is polarized.

45. A convertible head cover apparatus according to claim 29, wherein at least a portion of said transparent shield member deflects ultra violet light.

46. A convertible head cover apparatus according to claim 29, wherein said front flap in said extended position attaches flushly with each said flap member when each said flap member is in said extended position.

47. A convertible head cover apparatus according to claim 29, wherein each said flap member is adapted to substantially cover a temple of said lateral portion of said mammalian head when each said flap member is in said extended position.

48. A convertible head cover apparatus according to claim 29, wherein said front flap in said extended position is adapted to insulate a periphery of said face of said mammalian head.

49. A convertible head cover apparatus according to claim 29, wherein an insulating edging of said front flap mates with an insulating edging of each said flap member when each said flap member and said front flap are both in said extended positions and when each said flap member covers a lateral portion of said mammalian head.

50. A convertible head cover apparatus according to claim 29, wherein said front flap further comprises at least one tab, wherein said at least one tab removably attaches to each said flap member when each said flap member and said front flap are both in said extended positions and when each said flap member covers a lateral portion of said mammalian head.

51. A convertible head cover apparatus according to claim 29, wherein said at least one tab removably attaches to said crown member to maintain said front flap in said stowed position.

52. A convertible head cover apparatus according to claim 29, wherein said front flap in said stowed position is removably attached to said crown member.

53. A convertible head cover apparatus according to claim 29, wherein said front flap is removably attached to each said flap member when said front flap is in said extended position and when each said flap member in said extended position covers a lateral portion of said mammalian head.

54. A convertible head cover apparatus according to claim 29, wherein a first each said flap member is removably attached to a second each said flap member when said first and second flap members are in said extended position and when said first and second flap members each cover a lateral portion of said mammalian head.

55. A convertible head cover apparatus according to claim 29 wherein each said flap member is removably attached to a second flap member when each said flap member is in said stowed position and when each said flap member is adapted to cover a lateral portion of said mammalian head.

56. A convertible head cover apparatus, comprising:
a crown member adapted to be placed on a top portion of a crown of a mammalian head;
a first and second flap member pivotally attached to said crown member, said first and second flap member moveable from a stowed position adjacent said crown member to an extended position, said first and second flap member each adapted to cover a temple of said mammalian head when said first and second flap members are in said extended position;
a rear flap member pivotally attached to said crown member, said rear flap member moveable from a stowed position adjacent said crown member to an extended position, said rear flap member adapted to cover a posterior portion of said mammalian head in said extended position;
said crown member having a front flap, said front flap housing a transparent shield member, said front flap being pivotally attached to said crown member and moveable from a stowed position to an extended position, said front flap adapted to cover a portion of a face of said mammalian head when said front flap is in said secured extended position;
said transparent shield member flexing from approximately 1 to approximately 60 degrees when said front flap moves between said stowed position and said extended position.

57. A convertible head cover apparatus, comprising:
a crown member adapted to cover a top portion of a crown of a mammalian head;
at least one flap member pivotally attached to said crown member, each said flap member moveable from a stowed position adjacent said crown member to an extended position, each said flap member adapted to cover at least one of a lateral and posterior portion of said mammalian head in said extended position;
each said flap member independently attached to said crown member, each said flap member moveable between said stowed position and said extended position independent of movement of another flap member;
said crown member having a front flap, said front flap housing a transparent shield member, said front flap being pivotally attached to said crown member and moveable from a stowed position to an extended position, said front flap adapted to cover a portion of a face of said mammalian head when in said extended position;
said transparent shield member flexing when said front flap moves between said stowed position and said extended position.

58. A convertible head cover apparatus according to claim 57, wherein said transparent shield member flexes concavely when said front flap is moved to said extended position.

59. A convertible head cover apparatus according to claim 57, wherein said at least one flap member comprises two flap members, each said flap member having first and second opposed extended portions.

60. A convertible head cover apparatus according to claim 57, wherein said transparent shield member reverse-flexes when said front flap is moved to said stowed position.

61. A convertible head cover apparatus according to claim 57, wherein each said flap member comprises linearly variable attachment points for removable attachment of said front flap to each said flap member when said front flap and each said flap member is each in said extended position.

62. A convertible head cover apparatus according to claim 57, wherein said transparent shield member housed by said front flap being flexibly responsive to said linear positioning of said attachment points causes flexure of said transparent shield member.

63. A convertible head cover apparatus according to claim 57, wherein said transparent shield member flexes concavely to substantially conform to a shape of said face of said mammalian head when said front flap is removably attached to each said flap member when said front flap and each said flap member are each in said extended position.

64. A convertible head cover apparatus according to claim 57, wherein said transparent shield member reverse-flexes to conform to a shape of said crown member when said front flap is moved to said stowed position.

65. A convertible head cover apparatus according to claim 57, wherein each said flap member is attached to a lower rim of said crown member along a first predetermined length of said lower rim;
wherein said front flap is attached to said lower rim of said crown member along a second predetermined length of said lower rim;
wherein said first and second predetermined lengths of said lower rim comprise a complete length of said lower rim; and
wherein a combination of each said flap member and said front flap are adapted to cover a three hundred sixty degree horizontal circumferential extent of said mammalian head below said crown member.

66. A convertible head cover apparatus according to claim 57, wherein said transparent shield member is comprised of polyethylene.

67. A convertible head cover apparatus according to claim 57, wherein at least a portion of said transparent shield member is polarized.

68. A convertible head cover apparatus according to claim 57, wherein at least a portion of said transparent shield member deflects ultra violet light.

69. A convertible head cover apparatus according to claim 57, wherein said front flap in said extended position attaches flushly with each said flap member when each said flap member is in said extended position.

70. A convertible head cover apparatus according to claim 57, wherein each said flap member is adapted to substantially cover a temple of said lateral portion of said mammalian head when each said flap member is in said extended position.

71. A convertible head cover apparatus according to claim 57, wherein said front flap in said extended position is adapted to insulate a periphery of said face of said mammalian head.

72. A convertible head cover apparatus according to claim 57, wherein an insulating edging of said front flap mates with an insulating edging of each said flap member when each said flap member and said front flap are both in said extended positions and when each said flap member covers a lateral portion of said mammalian head.

73. A convertible head cover apparatus according to claim 57, wherein said front flap further comprises at least one tab, wherein said at least one tab removably attaches to each said flap member when each said flap member and said front flap are both in said extended positions and when each said flap member covers a lateral portion of said mammalian head.

74. A convertible head cover apparatus according to claim 73, wherein said at least one tab removably attaches to said crown member to maintain said front flap in said stowed position.

75. A convertible head cover apparatus according to claim 57, wherein said front flap in said stowed position is removably attached to said crown member.

76. A convertible head cover apparatus according to claim 57, wherein said front flap is removably attached to each said flap member when said front flap is in said extended position and when each said flap member in said extended position covers a lateral portion of said mammalian head.

77. A convertible head cover apparatus according to claim 57, wherein a first each said flap member is removably attached to a second each said flap member when said first and second flap members are in said extended position and when said first and second flap members both cover a lateral portion of said mammalian head.

78. A convertible head cover apparatus according to claim 57, wherein each said flap member is removably attached to a second flap member when each said flap member is in said stowed position and when each said flap member is adapted to cover a lateral portion of said mammalian head.

79. A convertible head cover apparatus, comprising:
a crown member adapted to be placed on a top portion of a crown of a mammalian head;
a first and second flap member pivotally attached to said crown member, said first and second flap member moveable from a stowed position adjacent said crown member to an extended position, said first and second flap member each adapted to cover a temple of said mammalian head when said first and second flap members are in said extended position;
a rear flap member pivotally attached to said crown member, said rear flap member moveable from a stowed position adjacent said crown member to an extended position, said rear flap member adapted to cover a posterior portion of said mammalian head in said extended position;
said crown member having a front flap, said front flap housing a transparent shield member, said front flap being pivotally attached to said crown member and moveable from a stowed position to an extended position, said front flap adapted to cover a portion of a face of said mammalian head when said front flap is in said secured extended position;
said transparent shield member flexing when said front flap moves between said stowed position and said extended position.
